(19) United States
(12) Patent Application Publication
(10) Pub. No.: US 2023/0416847 A1
(43) Pub. Date: Dec. 28, 2023

(54) SCREENING PLATFORMS

(71) Applicant: MAYO FOUNDATION FOR MEDICAL EDUCATION AND RESEARCH, Rochester, MN (US)

(72) Inventors: Marina R. WALTHER-ANTONIO, Rochester, MN (US); Yuguang LIU, Rochester, MN (US); Samuel P. KOUNAVES, Winchester, MA (US); Dongyu LIU, Medford, MA (US); Miles CLARK, Somerville, MA (US)

(21) Appl. No.: 18/028,394

(22) PCT Filed: Sep. 24, 2021
(86) PCT No.: PCT/US2021/051928

§ 371 Date: Mar. 24, 2023

(30) Foreign Application Priority Data
Sep. 24, 2020 (US) 63082815

(51) Int. Cl. C12Q 1/70; C12Q 1/689; B01L 3/00

(52) U.S. Cl. CPC C12Q 1/701; C12Q 1/705; C12Q 1/689; B01L 3/508; C12Q 1/6837

(57) ABSTRACT

Materials and methods are provided herein for determining if a subject has, or is at risk of developing, a clinical condition (e.g., cervical cancer). For example, this document provides a cost-effective self-test for determining whether a biological fluid from a subject contains a high-risk HPV strain, and a method for use of the self-test.

### CROSS-REFERENCE TO RELATED APPLICATIONS

This application claims priority from U.S. Provisional Application Ser. No. 63/082,815, filed Sep. 24, 2020. The disclosure of the prior application is considered part of (and is incorporated by reference in) the disclosure of this application.

### TECHNICAL FIELD

This document relates to materials and methods for determining if a subject has, or is at risk of developing, a clinical condition that can be detected based on the presence of particular markers. For example, this document relates to a rapid and cost-effective self-test for determining whether a biological fluid from the subject contains a nucleic acid or polypeptide marker for a virus (e.g., a high-risk HPV strain that may indicate cervical cancer or oropharyngeal cancer) or a bacterium (e.g., Escherichia or Salmonella).

### BACKGROUND

Many diagnostic assays for various clinical conditions, including assays that involve the use of polymerase chain reaction (PCR) or enzyme-linked immunosorbent assays (ELISAs), can take 24 to 48 hours or even longer to produce results, which can negatively impact urgent decision making in particularly vulnerable patients. For example, cervical cancer often is asymptomatic at an early and curative stage. Readily available and reliable screening is important, particularly in populations without access to human papillomavirus (HPV) vaccination. In addition, because the screening for cervical and oropharyngeal cancer is physically and psychologically uncomfortable and carriage of the virus has been linked to a history of multiple sexual partners and sexual practices, many individuals at risk fail to undergo appropriate screening. The performance of a single HPV testing round has been associated with a significant reduction in the number of advanced cervical cancers, and HPV testing has been demonstrated to be more sensitive for detecting cancerous and precancerous microlesions than visual inspection with acetic acid and cytologic testing (Sankaranarayanan et al., N Engl J Med 360(14):1385-1394, 2009). Approaches such as the CAREHPV® test (Qiagen Inc.; Valencia, CA) offer a sensitivity of 90% and specificity of 84% (Qiao et al., The Lancet Oncol 9(10):929-936, 2008) for detecting pre-malignant or malignant lesions, but the cost, processing time, and need for laboratory facilities may be prohibitive for using such approaches in low-resource settings. Further, cervical cancer screening in low-resource areas may be marginalized compared to acute medical problems, there may be social stigma related to HPV infection or even visiting a gynecologist in conservative communities, and the local healthcare infrastructure may not have the resources to support a women's health program.

### SUMMARY

The present document is based, at least in part, on the development of testing materials and methods that can expedite screening capabilities while providing accurate, rapid, and affordable diagnosis. For example, the materials and methods described herein provide a colorimetric diagnostic platform that is self-contained and can be performed and interpreted by a user in private, similarly to a pregnancy test. In some cases, the platform can perform as a substitute for Polymerase Chain Reaction (PCR)-based tests. The test can detect the presence of, for example, high-risk HPV strains in biological samples such as urine, vaginal secretions, and saliva, but the flexibility of the platform also allows for its adaption to other disease targets, such as bacterial or viral infections (e.g., by enteropathogenic and flu agents). For example, the materials and methods provided herein can be used to indicate that a mammal is infected with HPV or influenza virus, is a carrier of Streptococcus pneumoniae, or contains circulating cardiac endothelial cells (CECs), which can indicate cardiac emergency. Use of the materials and methods described herein (e.g., in self-testing or point-of-care testing) can lead to timely and informed medical intervention by eliminating delays in diagnostic testing, since there is no need to transport samples to a laboratory setting, and no need for specialized personnel to perform the required assays and interpret the results.

In some embodiments, for example, this document provides an inexpensive, reliable self-test for high-risk HPV. The screening test can be used to identify subjects (e.g., human females) having a high-risk carrier status, while minimizing barriers and providing an opportunity for early intervention before disease leads to significant morbidity and mortality. The self-test can be cost effective, easily distributed, and can be administered in the privacy of a user's home. This may lead to earlier detection of HPV, resulting in clinical follow up in a more timely manner.

In a first aspect, this document features an elongate support having at least a first surface, a first nucleic acid immobilized on the first surface, and a second nucleic acid reversibly attached to the first surface or to a second surface, where the first surface is silanized, where the first nucleic acid is complementary to a first segment of a selected nucleic acid marker, and where the second nucleic acid is complementary to a second segment of the selected nucleic acid marker. The elongate support can include glass. The elongate support can be a glass rod (e.g., a capillary tube). The first nucleic acid can be immobilized on the first surface via biotin-streptavidin coupling. The second nucleic acid can be dry-stored on the first surface or the second surface. The second nucleic acid can be coupled to a means for visual detection. The means for visual detection can be horseradish peroxidase (HRP). The marker can be from one or more high-risk human papillomavirus (HPV) strains. The one or more high-risk HPV strains can include one or more of HPV 16, HPV 18, HPV 31, HPV 33, HPV 35, HPV 39, HPV 45, HPV 51, HPV 52, HPV 56, HPV 58, HPV 59, and HPV 68. The marker is from an infectious agent (e.g., an influenza virus or Escherichia coli). The elongate support can have an external surface that includes a coating or handle. The silanized first surface can include functional groups derived from (3-aminopropyl)triethoxysilane (APTES), N-(2-amionethyl)-3-aminopropyltriethoxysilane (AEAPTES), N-(2-aminoethyl)-3-aminopropyltrimethoxy-silane (AEAPTMS), or N-(6-aminohexyl)amionmethyltriethoxysilane (AHAMTES). The silanized first surface can include benzaldehyde-protected silane groups.

In another aspect, this document features an elongate support having a silanized surface with a nucleic acid immobilized thereon, where the nucleic acid is complementary to a first segment of a selected nucleic acid marker. The elongate support can include glass. The elongate support can be a glass rod (e.g., a capillary tube). The nucleic acid can be immobilized on the silanized surface via biotin-streptavidin coupling. The marker can be from one or more high-risk HPV strains (e.g., one or more of HPV 16, HPV 18, HPV 31, HPV 33, HPV 35, HPV 39, HPV 45, HPV 51, HPV 52, HPV 56, HPV 58, HPV 59, and HPV 68). The marker can be from an infectious agent (e.g., an influenza virus or Escherichia coli). The elongate support can have an external surface that includes a coating or handle. The silanized surface can include functional groups derived from APTES, AEAPTES, AEAPTMS, or AHAMTES. The silanized surface can include benzaldehyde-protected silane groups.

In another aspect, this document features a kit that includes (a) an elongate support having a first surface, a first nucleic acid immobilized on the first surface, and a second nucleic acid reversibly attached to the first surface or to a second surface, where the first surface is silanized, where the first nucleic acid is complementary to a first segment of a selected nucleic acid marker, and where the second nucleic acid is complementary to a second segment of the selected nucleic acid marker and is labeled with a means for visual detection; (b) a receptacle for receiving a biological fluid sample; and (c) a vessel containing a substrate that interacts with the means for visual detection. The first nucleic acid can be immobilized on the first surface via biotin-streptavidin coupling. The second nucleic acid can be dry-stored on the first surface or the second surface. The means for visual detection can be HRP. The marker can be from one or more high-risk HPV strains (e.g., one or more of HPV 16, HPV 18, HPV 31, HPV 33, HPV 35, HPV 39, HPV 45, HPV 51, HPV 52, HPV 56, HPV 58, HPV 59, and HPV 68). The marker can be from an infectious agent (e.g., an influenza virus or Escherichia coli). The substrate can be tetramethylbenzidine (TMB). The elongate support can have an external surface that includes a coating or handle. The silanized surface can include functional groups derived from APTES, AEAPTES, AEAPTMS, or AHAMTES. The silanized first surface can include benzaldehyde-protected silane groups. The elongate support can include glass. The elongate support can be a glass rod (e.g., a glass capillary tube).

In another aspect, this document features a kit that includes (a) an elongate support having a silanized surface with a first nucleic acid immobilized thereon, where the first nucleic acid is complementary to a first segment of a selected nucleic acid marker; (b) a receptacle for receiving a biological fluid sample; (c) a first vessel containing a second nucleic acid that is complementary to a second segment of the selected nucleic acid marker, where the second nucleic acid is labeled with a means for visual detection; and (d) a second vessel containing a substrate that interacts with the means for visual detection. The first nucleic acid can be immobilized on the silanized surface via biotin-streptavidin coupling. The means for visual detection can be HRP. The marker can be from one or more high-risk HPV strains (e.g., one or more of HPV 16, HPV 18, HPV 31, HPV 33, HPV 35, HPV 39, HPV 45, HPV 51, HPV 52, HPV 56, HPV 58, HPV 59, and HPV 68). The marker can be from an infectious agent (e.g., an influenza virus or Escherichia coli). The first vessel can contains the second nucleic acid in a fluid that contains 2% polyethylene glycol (PEG). The substrate can be TMB. The elongate support can have an external surface with a coating or handle. The silanized surface can include functional groups derived from APTES, AEAPTES, AEAPTMS, or AHAMTES. The silanized surface can include benzaldehyde-protected silane groups. The elongate support can include glass. The elongate support can be a glass rod (e.g., a capillary tube).

In another aspect, this document features a method for determining that a biological fluid contains a selected marker, where the method includes (a) providing an elongate support having a first surface, a first nucleic acid immobilized on the first surface, and a second nucleic acid reversibly attached to the first surface or to a second surface, where the first surface is silanized, where the first nucleic acid is complementary to a first segment of a selected nucleic acid marker, and where the second nucleic acid is complementary to a second segment of the selected nucleic acid marker and is labeled with a means for visual detection; (b) placing at least a portion of the elongate support into a vessel containing a biological fluid, such that the first or first and second surfaces are contacted by the biological fluid; (c) removing the elongate support from the vessel and rinsing the elongate support; (d) placing at least a portion of the elongate support into a vessel containing a substrate that interacts with the means for visual detection, such that the first surface is contacted by the substrate; and (e) visually inspecting the vessel containing the substrate, or a sample of the substrate, to determine that the signal is present, thus indicating the presence of the selected marker in the biological fluid. The elongate support can include glass. The elongate support can be a glass rod (e.g., a capillary tube). The first nucleic acid can be immobilized on the first surface via biotin-streptavidin coupling. The second nucleic acid can be dry-stored on the first surface or the second surface. The second nucleic acid can be coupled to a means for visual detection (e.g., HRP). The marker can be from one or more high-risk HPV strains (e.g., one or more of HPV 16, HPV 18, HPV 31, HPV 33, HPV 35, HPV 39, HPV 45, HPV 51, HPV 52, HPV 56, HPV 58, HPV 59, and HPV 68). The marker can be from an infectious agent (e.g., an influenza virus or Escherichia coli). The elongate support can have an external surface with a coating or handle. The silanized first surface can include functional groups derived from APTES, AEAPTES, AEAPTMS, or AHAMTES. The silanized first surface can include benzaldehyde-protected silane groups.

In still another aspect, this document features a method for determining that a biological fluid contains a selected marker, where the method includes (a) providing an elongate support having a silanized surface with a first nucleic acid immobilized thereon, where the first nucleic acid is complementary to a first segment of a selected nucleic acid marker; (b) placing at least a portion of the elongate support into a first vessel containing a biological fluid, such that the silanized surface is contacted by the biological fluid; (c) removing the elongate support from the first vessel and rinsing the elongate support; (d) placing at least a portion of the elongate support into a second vessel containing a second nucleic acid that is complementary to a second segment of the selected nucleic acid marker and is labeled with a means for visual detection, such that the silanized surface is contacted by the second nucleic acid; (e) removing the elongate support from the second vessel and rinsing the elongate support; (f) placing at least a portion of the elongate support into a third vessel containing a substrate that interacts with the means for visual detection, such that the silanized surface is contacted by the substrate; and (g) visually inspecting the third vessel containing the substrate, or a sample of the substrate, to determine that the signal is present, thus indicating the presence of the selected marker in the biological fluid. The elongate support can include glass. The elongate support can be a glass rod (e.g., a capillary tube). The nucleic acid can be immobilized on the silanized surface via biotin-streptavidin coupling. The marker can be from one or more high-risk HPV strains (e.g., one or more of HPV 16, HPV 18, HPV 31, HPV 33, HPV 35, HPV 39, HPV 45, HPV 51, HPV 52, HPV 56, HPV 58, HPV 59, and HPV 68). The marker can be from an infectious agent (e.g., an influenza virus or Escherichia coli). The elongate support can have an external surface with a coating or handle. The silanized surface can include functional groups derived from APTES, AEAPTES, AEAPTMS, or AHAMTES. The silanized surface can include benzaldehyde-protected silane groups.

In addition, this document features a method for preparing a silanized glass surface, where the method includes reacting a glass surface with a silane-containing compound, coupling an amino group of the silane-containing compound to biotin-N-hydroxysuccinimide (biotin-NHS) to yield a silanized, biotinylated surface, and reacting the silanized, biotinylated surface with benzaldehyde.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic depicting representative components of a glass rod support for detecting a target molecule (e.g., an HPV nucleic acid) in a fluid test sample using single stranded nucleic acid probes.

FIGS. 2A and 2B are schematics depicting representative components and steps in an embodiment of a glass rod-based test for detecting a target molecule in a fluid test sample, as described herein.

FIG. 3 is a schematic depicting representative components of a glass rod support for detecting a target molecule in a fluid test sample using DNAzyme reagents.

FIG. 4 is a schematic depicting representative components and steps in a glass rod-based test for using a DNAzyme reagent to detect a target molecule in a fluid test sample.

FIGS. 5A-5C are images showing representative test results using patient urine with nucleic acid probes specific for HPV16. FIG. 5A shows the colorometric change observed in the urine of an HPV16 positive patient 10 minutes after test completion (4 replicates). FIG. 5B shows the absence of colorometric change observed in the urine of an HPV16 negative patient 10 minutes after test completion (4 replicates). FIG. 5C depicts the absence of colorometric change detected in the urine of an HPV16 negative patient 2 hours after test completion (4 replicates).

FIG. 6 is an image indicting the level of HRP activity for 1 μM HRP-DNA after storage with or without 2% EtOH for 4 months at 4° C. The sample with 2% EtOH is on the left and the control (without EtOH) is on the right, as indicated in the figure. Both the sample and control were kept in the refrigerator for about 4 months in 2% PEG 1×PBS buffer.

FIG. 7 is a diagram showing different functional groups on a glass surface. Top, unreacted (3-aminopropyl)triethoxysilane (APTES); middle, immobilized biotin; and bottom, benzaldehyde-protected unreacted groups.

FIG. 8 is a schematic depicting representative components and steps in an embodiment of an analytical methodology for detecting genetic targets. HRP: horseradish peroxidase; TMB: tetramethylbenzidine.

### DETAILED DESCRIPTION

This document provides materials and methods for making and using diagnostic tests with samples of bodily fluid (e.g., urine, sputum, blood, serum, or cerebrospinal fluid) suspected of containing a particular marker molecule (e.g., a nucleic acid such as an RNA or DNA, or a polypeptide or protein). This document also provides kits containing the tests, as well as methods of using the test materials to detect markers from selected targets, and to determine whether cells containing a selected marker are present in a biological sample. Markers that may be detected using these materials and methods include, without limitation, nucleic acids (e.g., DNA or RNA, including mutant or aberrantly expressed nucleic acids that provide a signature for a disease), peptides, polypeptides, antibodies or antibody fragments, virus particles, and bacteria.

In some cases, an existing PCR or ELISA-based test can be adapted to the platform described herein. The platform can provide a high clinical value for detecting acute illness in patients who would benefit from real-time testing and immediate administration of targeted treatment, although the platform also can be applied to any chronic condition for which there is, for example, a nucleic acid or protein marker. As compared to standard diagnostic procedures, the materials and methods provided herein typically are faster (providing real-time results) and more affordable, and allow for the possibility of self-administration.

In some embodiments, the assay platform provided herein can be used as a self-test to identify high-risk HPV carriers, in methods for determining whether a subject is a carrier of high-risk HPV. In women, high-risk types of HPV (such as types 16, 18, 31, and 45) can cause changes in the cells of the cervix that can be seen as abnormalities on a Pap test. Abnormal cervical cell changes may resolve on their own without treatment, but some untreated cervical cell changes can progress to serious abnormalities and may lead to cervical cancer over time if they are not treated. The screening tests described herein can provide for rapid and easy detection of the high-risk HPV in carriers. The tests can take advantage of target strain labeling and capturing capabilities in a receptacle (e.g., a glass vial), based on complementary HPV nucleic acid immobilization and dry reagent storage. The test can be read in real-time, without requiring laboratory facilities or personnel for the processing of results, which can address some of the main challenges with implementation in low-resource communities. The test is aimed at matching the clinical standard for HPV testing reliability, with a user-perceived level of difficulty for use and interpretation that is comparable to standard home pregnancy test use and reading.

In some cases, the screening kits provided herein include a glass support (e.g., a glass rod) as a nucleic acid diagnostic platform that can be used in a clinical setting or as a self-test and read in real-time by a user, with minimal instruction. The described approach for nucleic acid detection can include pre-immobilization of nucleic acid (e.g., RNA or DNA) sequences that are complementary to target nucleic acid sequences (e.g., high-risk HPV nucleic acid sequences) and dry storage of reagents on the support that allow for labeling and capture of target strains, and produce colorimetric results interpretable by the naked eye. In some embodiments of the test methods described herein, nucleic acids complementary to marker sequences (e.g., sequences from high-risk HPV strains) can be modified with horseradish peroxidase (HRP) and dry-stored in vials. When the complementary nucleic acids are contacted with a fluid containing an HPV marker nucleic acid (e.g., RNA or DNA) and a tetramethylbenzidine (TMB) substrate, the fluid can turn blue to indicate the presence of a high-risk HPV strain.

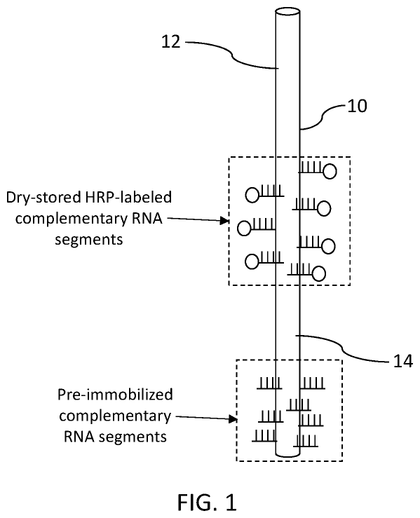

FIG. 1 depicts a representative support structure (illustrated as a rod) on which nucleic acids having a first sequence are immobilized and on which nucleic acids having a second sequence are dry stored. The support can be made from any appropriate material to which nucleic acids or other probes (e.g., biotin, streptavidin or other polypeptides, or antibodies) can be reversibly or permanently attached. Suitable materials include, without limitation, glass (e.g., borosilicate glass). The support can have any appropriate shape and dimensions. In some cases, for example, the support can be an elongate rod or tube (e.g., a glass capillary tube). In some cases, the support can have a proximal portion and a distal portion, and can have a length sufficient to allow a user to hold the support within the proximal portion so as not to contact nucleic acids that are attached (e.g., through immobilization or drying) more distally on the support.

When the support is a rod, for example, the rod can have any appropriate length. For example, a rod can have a length of about 10 mm to about 100 mm (e.g., about 10 to about 20 mm, about 20 to about 30 mm, about 30 to about 40 mm, about 40 to about 50 mm, about 50 to about 75 mm, or about 75 to about 100 mm). The rod can have any appropriate cross-sectional shape (e.g., circular or square), and a width or diameter of about 0.5 mm to about 2.5 mm (e.g., about 0.5 to about 0.7 mm, about 0.7 to about 1.0 mm, about 1.0 to about 1.2 mm, about 1.2 to about 1.4 mm, about 1.4 to about 1.6 mm, about 1.6 to about 1.8 mm, about 1.8 to about 2.0 mm, about 2.0 to about 2.2 mm, or about 2.2 to about 2.5 mm). When the rod is a capillary tube, the outer diameter can be, for example, about 0.5 to about 2.5 mm as above, and the inner diameter can be about 0.2 to about 2.3 mm (e.g., about 0.2 to about 0.5 mm, about 0.5 to about 0.7 mm, about 0.7 to about 1.0 mm, about 1.0 to about 1.2 mm, about 1.2 to about 1.4 mm, about 1.4 to about 1.6 mm, about 1.6 to about 1.8 mm, or about 1.8 to about 2.0 mm.

In some cases, the support can include a plurality of glass beads (e.g., glass microbeads). Using glass microbeads may provide an increased surface area for attachment of nucleic acids or other molecules, which in turn may enhance the capturing efficiency and increase hybridization or binding of targets. Glass beads can be easily dispersed in a fluid sample and readily mixed. After a suitable time to allow for binding of target molecules, the beads can be allowed to aggregate at the bottom of the fluid container due to gravity, such that a centrifuge is not required. It is noted, however, that centrifugation could be used to aggregate the beads. When the support includes glass beads, the beads can have any appropriate size. In some cases, for example, the beads can be about 1 mm or less in their largest dimension (e.g., about 500 μm to about 1 mm, about 100 μm to about 500 μm, about 50 μm to about 100 μm, or about 10 μm to about 50 μm in their largest dimension).

Nucleic acids attached to a support as described herein can have a length sufficient to allow for specific hybridization (e.g., 10-100 nucleotides, 15-75 nucleotides, nucleotides, 10-20 nucleotides, 20-30 nucleotides, 30-40 nucleotides, or 40-50 nucleotides). When a test is designed to look for nucleic acid from a particular target (e.g., a high risk strain of HPV), the support can have first and second nucleic acid sequences attached thereto. The first and second nucleic acid sequences can be complementary to first and second regions within the sequence of the target nucleic acid, where the first and second nucleic acid sequences are separate and non-overlapping (e.g., adjacent to one another or separated by a spacer) such that they can both hybridize to the target (e.g., to adjacent sequences within the target or to sequences within the target that are separated by a spacer) at the same time.

It is noted that the affinity of a capture nucleic acid for its target can be affected by the length of the capture nucleic acid, and suitable capture nucleic acid density is based on both affinity and accessibility. In some cases, to enhance affinity without extending the length of a capture nucleic acid, DNA/locked nucleic acid (DNA/LNA) chimeras can be used, as they can have increased stability as compared to DNA. See, e.g., Klamp et al., Sci Rep 3:1852, 2013. Locked nucleic acids (LNAs), often referred to as inaccessible RNA, are modified RNA oligonucleotides in which the ribose moiety is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in an oligonucleotide, and can hybridize with DNA or RNA according to Watson-Crick base-pairing rules.

Any suitable nucleic acid sequence from any appropriate target can be attached to a support as provided herein. For example, nucleic acid sequences for numerous HPV strains are known. Sequences that are conserved between high-risk HPV strains but are not found within low-risk HPV strains can be particularly useful. In some cases, qPCR probe sequences can be that are established and validated as described elsewhere can be useful. Representative examples of high- and low-risk HPV strains and their GENBANK® accession numbers include the following:
HPV 16 (high-risk): Accession No. 571514 (e.g., version 571514.1)
HPV 18 (high-risk): Accession No. KY502186 (e.g., version KY502186.1)
HPV 31 (high-risk): Accession No. JQ693766 (e.g., version JQ693766.1)
HPV 33 (high-risk): Accession No. KF536963 (e.g., version KF536963.1)
HPV 39 (high-risk): Accession No. A26661 (e.g., version A26661.1)
HPV 45 (high-risk): Accession No. AJ242956 (e.g., version AJ242956.1)
HPV 51 (high-risk): Accession No. KT725857 (e.g., version KT725857.1)
HPV 6 (low-risk): Accession No. S73503 (e.g., version S73503.1)
HPV 11 (low-risk): Accession No. U55993 (e.g., version U55993.1)
HPV 42 (low-risk): Accession No. A28090 (e.g., version A28090.1)
   Other examples of targets include, without limitation, nucleic acids and polypeptides from influenza viruses, the SARS-CoV-2 virus, Streptococcus pneumoniae, other bacteria, and cardiac endothelial cells.

It is to be noted that molecules other than nucleic acids can be attached to a support for use as probes and/or capture molecules. In some cases, for example, if the target is a polypeptide, an antibody or ligand that binds to the target polypeptide can be reversibly attached to the support, and an antibody that recognizes the resulting antibody-polypeptide complex or ligand-polypeptide complex can be immobilized on the support.

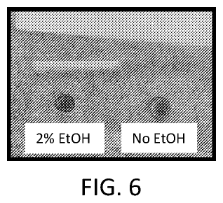

Any suitable method can be used to attach one or more probes (e.g., nucleic acids) to the support. In some cases, for example, one or more first nucleic acid (e.g., RNA or single stranded DNA) sequences can be immobilized on a silanized glass support surface (a glass support to which alkoxysilane molecules have been attached) via streptavidin-biotin interactions. In such cases, streptavidin can first be attached to the silane molecules on the surface of the support, and biotin-conjugated nucleic acid sequences can then be attached to the streptavidin. Any appropriate silanization agent can be attached to a glass surface. In addition to APTES (FIG. 6), silanization agents such as N-(2-aminoethyl)-3-amino propyltriethoxysilane (AEAPTES), N-(2-aminoethyl)-3-aminopropyltrimethoxysilane (AEAPTMS), and N-(6-aminohexyl)aminomethyltriethoxysilane (AHAMTES) can be used.

A benefit of using a capillary tube is that background signal can be minimized while still providing enough surface area to capture a sufficient amount of target DNA. In some cases, however, it may be useful to use a support with more surface area (e.g., a glass slide or glass tube) to which capture nucleic acids are attached even though the background signal caused by HRP-DNA interaction would likely be increased. Given a low level of target DNA in a sample, a higher background signal caused by a larger surface area could increase the detection limit significantly. Thus, systems and methods utilizing a larger surface area can be modified to reduce the background signal. In some cases, for example, a sample can be mixed (e.g., by stirring or vortexing) then waiting for the hybridization to complete. In some cases, a blocking buffer can be used to effectively reduce or eliminate nonspecific absorption from HRP. Nonspecific absorption from DNA (likely due to interaction between negatively charged DNA and positively charged APTES amine groups on the glass surface) can be harder to avoid. See, e.g., Aissaoui et al., Langmuir, 28(1):656-665, 2011; and Wu et al., Langmuir, 27(6):2731-2738, 2011. Such nonspecific absorption can be addressed, in some cases, by coating the glass surface with a layer of either hydrophobic or negatively charged organic functional groups (e.g., benzaldehyde as depicted in FIG. 6, or 4-(trifluoromethyl) benzaldehyde). To reduce potential loss of the coating due to the hydrolysis of the APTES layer (Asenath Smith and Chen, Langmuir, 24(21):12405-12409, 2008) the reaction time can be reduced in order to shorten the exposure time to water.

Any suitable method can be used to evaluate DNA attachment or hybridization to the support. Suitable methods include, without limitation, using Nanodrop to measure the remaining DNA or using customized HRP-DNA, which relies on additional DNA hybridization, or using Surface Plasmon Resonance (SPR) spectroscopy (Peterson et al., Nucl Acids Res, 29(24):5163-5168, 2001).

In addition to the immobilized nucleic acid, one or more HRP-modified second nucleic acid (e.g., RNA or single stranded DNA) sequences that are complementary to different nucleic acid sequences from the one or more HPV strains and are HRP-modified can be dry-stored on the surface of elongate support 10. The one or more second nucleic acid sequences can be immobilized on the same (or an overlapping) portion of the support as the first nucleic acid sequence, or can be immobilized on a portion of the support that is separate from the portion to which the first nucleic acid sequence is reversibly attached. In some cases, for example, the one or more second nucleic acids can be immobilized on distal portion 14 or on a more central portion of support 10, as depicted in FIG. 1. Any suitable method can be used to reversibly attach the second complementary nucleic acid(s) to the support, including methods known in the art (see, e.g., Sankaranarayanan et al., supra). In some embodiments, for example, HRP-modified nucleic acid sequences that are complementary to sequences from one or more high-risk HPV strains can be vacuum dry-stored (Ramachandran et al., Analyst 139(6):1456-1462, 2014) in sugar alcohol matrices (e.g., sucrose, trehalose, or polyvinyl alcohol (PVA) matrices) on the support to preserve their stability (see, e.g., Stevens et al., Lab on a Chip 8(12):2038-2045, 2008; and Ivanova and Kuzmina, Mol Ecol Resources 13(5):890-898, 2013). Proximal portion 12 of support 10 can be left free of nucleic acids, and can provide a location for a user to grasp and manipulate support 10 during use.

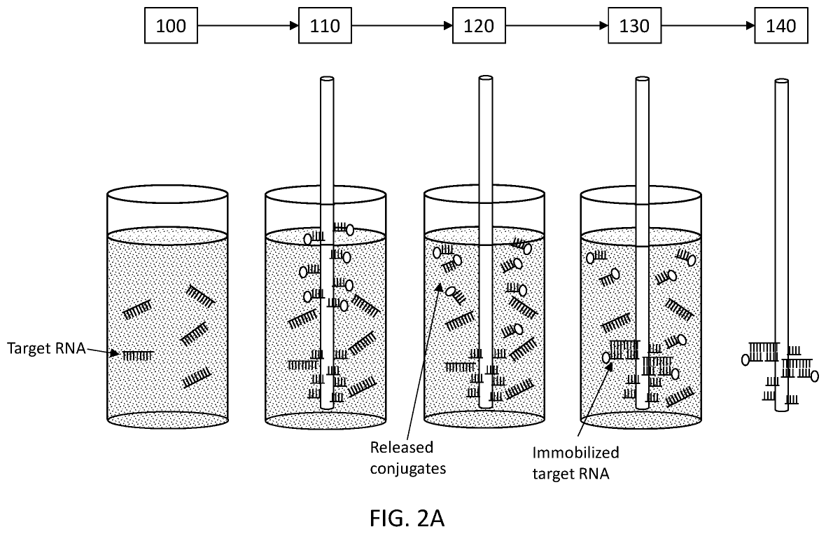

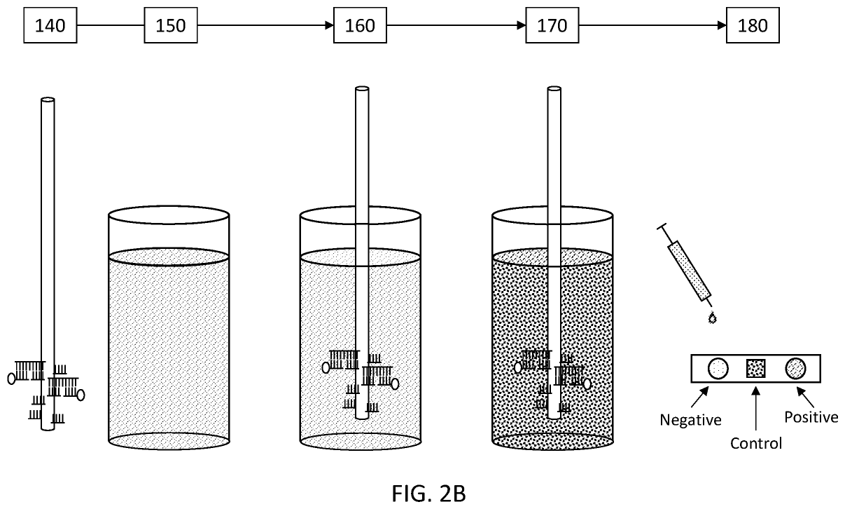

FIGS. 2A and 2B depict representative components and methodology for using an exemplary screening test kit as provided herein. The test relies on labeling and capture of target nucleic acid from a biological fluid sample (e.g., vaginal fluid or urine) in a receptacle (e.g., a 10 mL glass vial), followed by a wash step and a developing step. In the depicted method, elongate support 10 is provided on which one or more first nucleic acid (e.g., RNA) sequences that are complementary to HPV nucleic acid sequences (e.g., RNA sequences from one or more high-risk HPV strains) are immobilized (e.g., on distal portion 14 of support 10), as depicted in FIGS. 1 and 2A. The elongate support with the dry-stored and immobilized nucleic acids and a receptacle containing a bodily fluid (e.g., urine or vaginal fluid that is either undiluted or diluted with water, for example) as depicted in item 100 of FIG. 2A can be provided. As shown in item 110 of FIG. 2A, the portion of support 10 to which the nucleic acids are attached can be placed into the receptacle. This can cause the dry-stored, HRP-labeled complementary nucleic acids to be released from the support, as depicted in item 120 of FIG. 2A. The released HRP-coupled complementary nucleic acids can then bind to nucleic acids from target HPV strains (e.g., high-risk HPV strains) that are present in the sample, and the labeled target nucleic acids in the sample can be captured by the first complementary nucleic acid segments that are immobilized on the support, as depicted in item 130 of FIG. 2A. After the target nucleic acids are captured on the support, the support can be removed from the sample receptacle (item 140 of FIGS. 2A and 2B) and washed (Redon et al., DNA Microarrays for Biomedical Research: Methods and Protocols 267-278, 2009). The portion of support 10 to which the nucleic acids are attached can then be placed into a vessel containing a suitable amount of a highly sensitive substrate for HRP such as TMB, as shown in items 150 and 160 of FIG. 2B. Oxidation of TMB by HRP present in the vessel yields a blue color (illustrated by item 170 of FIG. 2B), indicating the capture of target RNA from an HPV strain that matches the first and second complementary nucleic acids. To interpret the results with the naked eye, a sample (e.g., a drop) of fluid from the vessel can be transferred onto a support (e.g., a paper strip) integrated with one or more control colors (e.g., a positive control color with or without a negative control color), as depicted in item 180 of FIG. 2B. The detection of a test color with an intensity similar to or darker than the positive control can serve as a positive reading, indicating the presence of a target HPV strain.

In some cases, blocking oligonucleotides can be used to help prevent denatured target DNAs from renaturing to itself. Blocking oligonucleotides can be selected to block renaturation while maintaining or, in some cases, increasing accessibility of the binding site of the target strain such that it can be captured or labeled, avoiding the kinetically stable hybridization caused by rapid denature-renature processes. For example, a blocking oligonucleotide can bind a target DNA adjacent to the sequence to which a capture nucleic acid probe can bind, maintaining accessibility of the target sequence for probes and preventing re-hybridization during annealing. A blocking oligonucleotide can have any appropriate length. In some cases, for example, a blocking oligonucleotide can have a length between about 10 and about 30 nucleotides (e.g., about 10 to 15 nucleotides, about 15 to 20 nucleotides, about 20 to 25 nucleotides, or about 25 to 30 nucleotides).

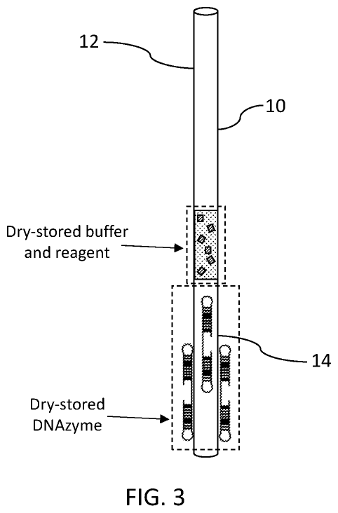

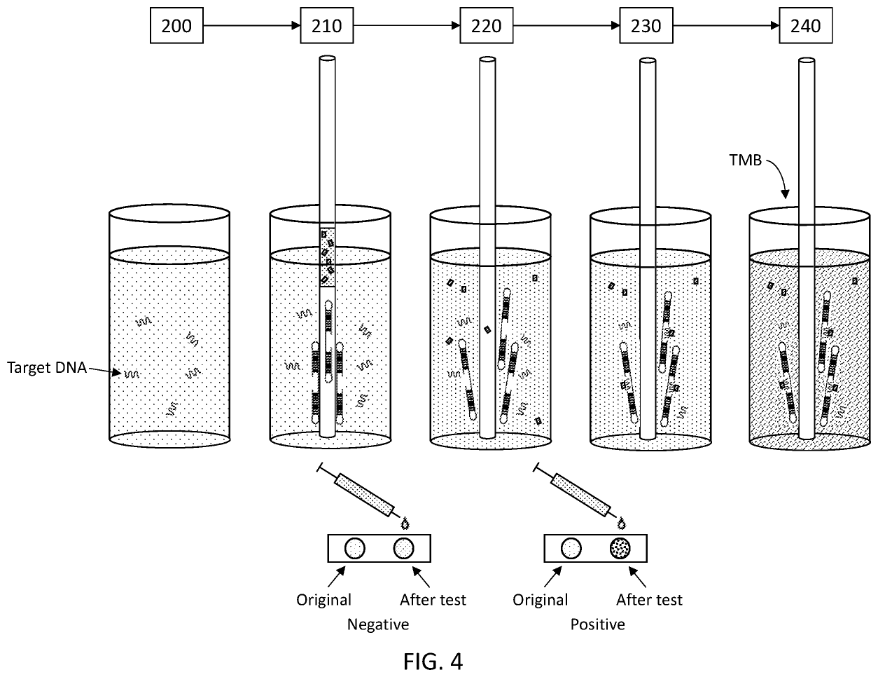

Other mechanisms also can be used to capture target markers and subsequently detect binding. In some cases, for example, a test can combine on-support dry reagent storage and the use of DNAzyme (an artificial catalytic DNA; Silverman, Chem Commun 3467-3485, 2008) capable of detecting targets such as nucleic acids and molecules secreted by cells (see, e.g., Zhou et al., Biosensors Bioelectronics 55:220-224, 2014; Wang et al., J Am Chem Soc 134:5504-5507, 2012; and Ali et al., Angewandte Chemie Int Ed 50:3751-3754, 2011). This approach typically is label-free, which can significantly reduce necessary user steps. DNAzyme and reagents required for the assays, such as a lysis buffer containing, for example, one or more enzymes (e.g., lysozyme, mutanolysin, and/or lysostaphin); one or more alkaline components (e.g., sodium hydroxide); and one or more surfactants (e.g., sodium dodecyl (lauryl) sulfate (SDS); TWEEN® (a polysorbate-type nonionic surfactant formed by ethoxylation of sorbitan before the addition of lauric acid; PLURONIC® (nonionic triblock copolymers composed of a central hydrophobic chain of polyoxypropylene (poly(propylene oxide)) flanked by two hydrophilic chains of polyoxyethylene (poly(ethylene oxide)); TRITON™ X-100 (a nonionic surfactant having the formula C₁₄H₂₂O(C₂H₄O)ₙ(n=9 or 10), with a hydrophilic polyethylene oxide chain and an aromatic hydrocarbon (4-phenyl) lipophilic or hydrophobic group; or any other reagent that can enable the release of genomic materials from the cells), can be concentrated and dry-stored in a sugar matrix (Ramachandran et al., Analyst 139:1456-1462, 2014) on a support, as depicted in FIG. 3. When the support is placed into a receptacle containing a bodily fluid sample (e.g., urine or vaginal fluid) (items 200 and 210 of FIG. 4), the DNAzyme and reagent(s) can be rehydrated and released (item 220 of FIG. 4), such that cells in the fluid are lysed to release their internal molecules. The released DNAzyme can bind to target molecules, whereupon a reagent (e.g., hemin6) released from the support into the solution can specifically attach to the DNAzyme-target conjugates as depicted in step 230 of FIG. 4. A highly sensitive substrate, such as TMB, can then be added (item 240 of FIG. 4), and the DNAzyme conjugates can cause a catalytic reaction that leads to a colorimetric shift toward blue, which can be discernable by the naked eye. In some cases, a sample (e.g., a drop) of fluid from the vessel can be transferred onto a support (e.g., a paper strip) integrated with one or more control colors (e.g., a negative control color as depicted in the lower portion of FIG. 4). The observation of a test color with an intensity similar to a negative control can serve as a negative reading, while the observation of a test color with an intensity similar to or darker than a positive control can serve as a positive reading, indicating the presence of a target HPV strain.

DNAzyme constructs can be prepared as described elsewhere (see, e.g., Kang et al., Nature Commun 5:5427, DOI: 10.1038/ncomms6427, 2014). In some embodiments, a DNAzyme construct can include a fluorogenic substrate (e.g., 5′-ACTCTTCCTAGCF-rA-QGGTTCGATCAAGA-3; SEQ ID NO:1, where (“F” indicates fluorescein-dT, “rA” indicates riboadenosine, and “Q” indicates dabcyl-dT), and a catalytic sequence (e.g., 5′-CACGGATCCTGACAAGGATGTGTGCGTTGTCGAGACCTGCGACCGGAACACTACACTGTGTGGGATGGATTTCTTTACAGTTGTGTGCAGCTCCGTCCG-3; SEQ ID NO:2). In some embodiments, where a DNAzyme is generated for an HPV 16 target, the fluorogenic substrate and the catalytic sequence can be covalently joined (e.g., through template-mediated enzymatic ligation) using a template marker sequence such as 5′- CACAGGGACATAATAATGGCATTTGTTGGGGTAACCAACTATTTGTTACTGTTGTTGATACTACACGCAGTACAAATATGTCATTATGTGCTGCCATATCTACTTCAGAAACTACATATAAAAATACTAACTTTAAGGAGTACCTACGACATGGGGAGGAATATGATTTACAGTTTATTTTTCAACTGTGCAAAATAACCTTAACTGCAGACGTTATGACATACATACA TTCTATGAATTCCACTATTTTGGAGGACTGGAATTTTGGTCTACAACCTCCCCCAGGAGGCACACTAGAAGATACTTATAGGTTTGTAACCCAGGCAATTGCTTGTCAAAAACATACACCTCCAGCACCTAAAGAAGATGATCCCCTTAAAAAATACACTTTTTGGGAAGTAAATTTAAAGGAAAAGTTTTCTGCAGACCTAGATCAGTTTCCTTTTGGACG-3′ (SEQ ID NO:3), where the template marker sequence of SEQ ID NO:3 is a target-specific sequence for HPV 16. The template marker sequence can be replaced with any other sequence to be specific for a different marker (e.g., a marker for a different HPV type, another virus, a bacterium, or a mammalian cell).

This concept can be tailored to cater to different clinical needs. For instance, when identifying low-abundance targets from complex clinical samples, a DNAzyme and its relevant chemistry can be designed to generate a fluorescent signal (Ali et al., supra; and Kang et al., Nature Commun 5:5427, 2014) that offers a higher resolution. In some embodiments, the fluorescent signal can be detected by a smart phone with a particular lens and filter attached to its camera (Zhu et al., Analyst 137:2541-2544, 2012). With an image analysis smart phone app, a user can obtain results with just a few taps on the screen.

The tests described herein can be used for detection of a wide variety of viral, bacterial, and cellular markers, and can be used with any bodily fluid sample (e.g., urine, sputum, blood, plasma, serum, cerebrospinal fluid, lymph fluid, or synovial fluid) that may contain free-floating virus particles, bacteria, or other cells of interest, for example. These materials and methods can have a high clinical value since they can be used to detect acute agents (e.g., acute viral agents) in vulnerable subjects who would benefit from real-time testing and immediate administration of targeted treatment.

In some cases, the tests provided herein can be adapted to detect various viral genetic signatures, including genetic signatures that indicate resistance to antiviral treatments. The materials and methods also can be used to monitor the status of infected patients. In some cases, for example, the materials and methods provided herein can be used to detect viral agents linked to respiratory infections. Real-time, rapid detection of viral agents that cause respiratory infection can be useful to determine whether immediate treatment should be pursued, particularly for vulnerable patients such as infants, the elderly, or those who are immunocompromised. Typical tests for influenza, respiratory syncytial virus (RSV), and severe acute respiratory syndrome coronavirus 2 (SARS-CoV-2) can require 24 hours or longer to retrieve a result. While this time frame may be acceptable for healthy adults, members of more vulnerable populations may require hospitalization in case life-threatening complications develop during the time period before results are obtained. In addition, people identified as being infected with a highly contagious virus such as SARS-Cov-2 should quarantine in order to reduce the risk of infecting others. The real-time test provided herein can allow the decision whether to hospitalize or quarantine to be made immediately, and in some cases, targeted treatment can be administered at the same clinical visit. Moreover, the assayed markers also can include one or more genes that confer resistance to known antiviral treatments, saving time in attempting a treatment stream that will fail.

In some cases, a test for detecting influenza, RSV, or SARS-CoV-2 can, in some embodiments, be similar to that for detecting HPV, except that instead of urine or cervical fluid, sputum can be used as the biological fluid sample. The sputum can be directly expelled by the subject into a vial, and an elongate support coupled to immobilized and dry-stored nucleic acids complementary to one or more virus markers can be dipped into the sample and processed as depicted in FIGS. 2A and 2B, for example.

Other viral agents can be screened for in blood samples using the methods and materials provided herein. These include, without limitation, hepatitis A, B, and C (e.g., to determine treatment for acute hepatitis), herpes simplex virus (e.g., to determine treatment of aseptic meningitis), cytomegalovirus (e.g., to determine treatment for infectious mononucleosis), human immunodeficiency virus (HIV) (e.g., for post-exposure prophylactic monitoring), rabies (e.g., to determine treatment for encephalitis), and varicella-zoster virus (e.g., to determine treatment for chickenpox).

In addition, the tests provided herein can be used to assay for the presence of bacterial agents in a biological fluid sample. For example, the disclosed materials and methods can be used to detect bacterial endotoxins and/or exotoxins, including those produced by members of the Escherichia, Salmonella, Shigella, Pseudomonas, Borderella, and Vibrio genera, which often are responsible for acute illness [e.g., enterotoxigenic E. coli (ETEC), which are one of the most common infectious agents causing acute gastroenteritis]. Nucleic acid probes also can be used to detect the genetic signatures of bacterial agents, but in some cases, lipopolysaccharide (LPS) probes linked to HRP can be used to detect the LPS signatures of bacterial agents. As for methods that involve detecting a genetic signature, an added substrate (e.g., TMB) can generate a visible signal if the matching LPS signature is present in the test sample. It is noted that methods utilizing LPS probes would likely not include using SDS to facilitate cell lysis.

Examples of ETEC targets include, without limitation, heat-stable toxins (STh and STp), heat-labile toxin (LT), and colonization factor antigens [CFs; also referred to as coli surface (CS) antigens], as well as Qnr genes (qnrA, qnrB, and/or qnrS). Representative sequences that may be targeted include those listed in TABLE 1. It is to be noted that these are exemplary sequences, and are not limiting.

TABLE 1

| Virulence | |
|---|---|
| Factor | Sequence (5′→3′) |
| LT | ACGGCGTTACTATCCTCTC (SEQ ID NO: 4) |
| STp | TCTTTCCCCTCTTTTAGTCAG (SEQ ID NO: 5) |
| STh | TTCACCTTTCCCTCAGGATG (SEQ ID NO: 6) |
| CS1 | TCCGTTCGGCTAAGTCAGTT (SEQ ID NO: 7) |
| CS4 | ACCTGCGGCAAGTCGTTT (SEQ ID NO: 8) |
| CS7 | CGCCGGTTACACGTAGTGAT (SEQ ID NO: 9) |
| CS12 | CCAGTCTATGCCAGGTTGCT (SEQ ID NO: 10) |
| CS3 | CTAGCTTTGCCACCACCATT (SEQ ID NO: 11) |
| CS21 | TCATGAGCCTGCTGGAAGTTATCA |
| | (SEQ ID NO: 12) |
| CS2 | AGTGGTGGCAGCGAAACTAT (SEQ ID NO: 13) |
| CS5 | TCCGCTCCCGTTACTCAG (SEQ ID NO: 14) |
| CFA/I | GCTTATTCTCCCGCATCAAA (SEQ ID NO: 15) |
| CS17 | GGAGACGCTGAATACAACTGA (SEQ ID NO: 16) |
| CS17/19 | CGGTGCGTTTAACACAGCTA (SEQ ID NO: 17) |
| CS8 | ATCCGGATTATCAAGCTCCA (SEQ ID NO: 18) |
| CS6 | CTGTGAATCCAGTTTCGGGT (SEQ ID NO: 19) |
| CS15 | CGAAATTGGACAAGCGATG (SEQ ID NO: 20) |
| CS20 | AGGTATCCAAATCCGCACTG (SEQ ID NO: 21) |
| CS18 | ATCCGTCAGGTGTTTGTGGT (SEQ ID NO: 22) |
| CS13 | GGGACTGCCACAATGAATTT (SEQ ID NO: 23) |
| CS14 | TTTGCAACCGACATCTACCA (SEQ ID NO: 24) |
| CS22 | ATTGGACAAGCGTCCAACAC (SEQ ID NO: 25) |
| qnrA | AGAGGATTTCTCACGCCAGG (SEQ ID NO: 26) |
| qnrB | GGMATHGAAATTCGCCACTG (SEQ ID NO: 27) |
| qnrS | GCAAGTTCATTGAACAGGGT (SEQ ID NO: 28) |

In further embodiments, the materials and methods provided herein can be used to detect bacteria or other cells (e.g., eukaryotic cells) directly. For assays designed to detect bacteria, for example, SDS can be used to lyse at least some of the bacterial cells, the lysate can be placed in a vial, and a support coupled to nucleic acids with sequences complementary to, for example, the 16S RNA of the target bacteria have been dry-stored and immobilized can be dipped into the vial. Bacterial detection using DNAzyme-based methods may not require dry-storage of anything other than the DNAzyme, since bacterial lysates can specifically react with an immobilized DNAzyme designed for the target bacteria. Methods of detecting eukaryotic (e.g., endothelial) cells can utilize a support on which DNA complementary to a sequence from the target cells has been immobilized. The rest of the procedure and detection mechanism can be carried out as described herein for bacteria or virus particles.

The methods and materials described herein also can utilize immunoglobulins of various types (e.g., IgA, IgD, IgE, IgG, and IgM) for real-time testing of exposure to pathogens, immunization status, and allergens. For such applications, the SDS used for cell lysis may be replaced with a detergent such as TRITON® X-100 or TWEEN®, for example. Antibodies (e.g., anti-IgA, anti-IgD, anti-IgE, anti-IgG, or anti-IgM antibodies), can be immobilized on a support using, for example, a method that relies on a condensation reaction between an aldehyde group on the antibodies and the hydrazide group on the modified glass surface, as described elsewhere (Gering et al., J Colloid Interface Sci 252(1):50-55, 2002). HRP-functionalized secondary antibodies also can be dry-stored in a sugar matrix on the support. The portion of the support containing the antibodies and secondary antibodies can be inserted into a fluid sample containing target molecules, such that the target molecules can be captured by the pre-immobilized antibodies and the dry-stored, HRP-functionalized antibodies can be rehydrated by the fluid and released from the sugar matrix. The target molecules then can become sandwiched between the pre-immobilized antibodies and the HRP-functionalized antibodies. At this point, the support can be removed from the sample, washed (e.g., three times), and placed into a vessel containing the TMB substrate.

In addition, the materials and methods provided herein can be used to test for free-floating genetic markers in blood or another bodily fluid, expanding liquid biopsies to real-time application. For such methods, first and second nucleic acid probes, or a nucleic acid probe such as a DNAzyme and a reagent that can specifically bind to the DNAzyme when it is recognized by the genetic material of interest, can be positioned on a support (e.g., where a first nucleic acid is immobilized on the support surface and a second, HRP labeled nucleic acid is reversibly attached to the support surface), without a reagent for lysing cells. If the genetic marker of interest is present within a sample, it can bind to the first and second nucleic acid probes when the support is placed into the sample, resulting in generation of a signal when the support is washed and contacted by the TMB substrate, thus indicating a positive result.

The materials described can be included in kits for detecting selected markers of, for example, particular viruses or bacteria. A kit can include an elongate support having one or more nucleic acids, polypeptides, LPS or other probes reversibly and/or permanently immobilized thereon. In some cases, a kit can include an elongate support having a nucleic acid probe and a reagent reversibly immobilized thereon. A kit also can include one or more receptacles for biological fluids (e.g., urine, vaginal fluid, or sputum). In general, a receptacle can be large enough to contain a typical sample of body fluid obtained from a subject, such as a 0.5 to 10 mL (e.g., 0.5 to 1 mL, 1 to 3 mL, 3 to 5 mL, 5 to 10 mL, 0.5 mL, 1 mL, 2 mL, 2.5 mL, 3 mL, 5 mL, 7.5 mL, or 10 mL) sample of urine or vaginal fluid, for example. A kit also can include a vessel (e.g., a vial or tube) containing a suitable amount (e.g., 0.5 to 10 mL, 1 to 5 mL, 5 to 10 mL, 0.5 mL, 1 mL, 5 mL, or 10 mL) of TMB or another detectable substrate. In some cases, the test kits provided herein can utilize commercially available glass vials (e.g., 5 to 10 mL, 10 to 20 mL, or 20 to 50 mL glass vials), which can largely reduce the need for microfabrication as compared with other point-of-care diagnostic devices.

In some cases, a kit can include a control component having a support with a positive control, a negative control, or both. The control component support also can include a portion for receiving a test sample, which may facilitate comparison to the positive and/or negative controls. The support can be, for example, a test paper strip. In some cases, the control (e.g., the test paper strip) included with the kits provided herein and integrated with the control(s) can be calibrated to compensate for background noise, facilitating interpretation of the result.

The kits provided herein can use DNA or proteins as targets for specific detection. Materials and methods as described herein can significantly lower the background signal, thus enabling the use of ELISA methods to detect biomarkers for diseases such as HPV, even at extremely low concentrations in bodily fluids. The kits provided herein therefore are robust and user friendly, such that a person with no experience in biochemistry can use them. This can be especially useful for quick and simple field diagnostic kits where delicate instruments and expert personal are not available. These materials and methods also can enable ELISA to become a cheap alternative for disease diagnosis where it was previously unpractical due to the high background signal and potential false positive readings.

The invention will be further described in the following examples, which do not limit the scope of the invention described in the claims.

### EXAMPLES

### Example 1—HPV Testing using Functionalized Vials

A test platform was developed for high-risk HPV strains (e.g., HPV types 16, 18, 31, 33, 35, 39, 45, 51, 52, 56, 58, 59, and/or 68) and low risk HPV strains (e.g., HPV types 6, 11, 40, 42, 43, 44, 53, 54, 61, 72, 73, and/or 81; de Sanjose et al. (2010) The Lancet Oncol 11(11):1048-1056) added to sterile saline as substrates at variable concentrations consistent with what would be expected in a biological sample (<1 femtomole, 600,000,000 copies (Denny et al. (2005) JAMA 294(17):2173-2181). The sensitivity and specificity of the test were calculated and adjusted until the levels displayed by the clinical standard test were reached, demonstrating efficacy and performance of the low-cost platform as compared to standard of care tests.

In particular, silanized clear glass vials (20 mL, Thermo Fisher Scientific) were obtained. One percent (1%) agarose in purified water was poured into the vial at 70° C., such that the bottom interior surface of the vial was covered. After agarose gelling, the vial was dried in air. 20 mM NaIO₄ was prepared under suitable conditions (e.g., in a chemical hood that could vent hazardous gases). The NaIO₄ solution was added into the agarose-coated vials at room temperature for 30 minutes for agarose activation. The activation led to the formation of aldehyde groups in the agarose, enabling the covalent binding of amino groups. DNA was suspended in spotting buffer (0.15 M NaCl, 0.1 M NaHCO₃, pH8.5), and pipetted onto the agarose film in the vial. The vials were incubated in a humid incubator overnight and dried at room temperature. Drops of sodium borohydride solution (50 mg NaBH₄ in 30 mL Phosphate Buffer Saline (PBS) with 10 mL ethanol) were pipetted into the vials under suitable conditions (e.g., in a chemical hood). After five minutes, the vials were washed three times with 0.2% sodium dodecyl sulfate (SDS) for 2 minutes, then washed twice in distilled water for 1 minute, and dried at room temperature.

Amino-modified complementary DNA segments were immobilized on the activated agarose film via NH₂ groups, and the vials were dried at room temperature. Horseradish peroxidase (HRP)-modified DNA segments were added to sugar alcohol matrices (sucrose, trehalose, polyvinyl alcohol (PVA)) to preserve their stability, and then added into the vials, which were then vacuum-dried at 30° C. for 2 hours. The vials were stored in a dry place at room temperature.

### Example 2—HPV Testing using Dip and Read Capillaries

A biotin—streptavidin bond was used to attach DNA probes to a silanized glass surface on glass capillary tubes. Although the biotin-streptavidin system does not provide a covalent bond, it is robust, stable, and irreversible under a wide variety of conditions. Initial tests with this system were conducted using streptavidin-modified glass slides, followed by glass vials. However, irreproducibility between the glass slides and the vials was observed, perhaps caused by poor rinsing of the vials due to their mouth size. After further testing, it was decided to use glass melting-point capillaries instead of glass vials. The capillaries were narrow glass tubes with rounded ends.

While transitioning to capillary tubes and determining the logistics of their fabrication, work was conducted on glass slides to identify ways to increase signal and reduce background. For example, due to interactions between immobilized streptavidin and HRP, some HRP was retained on the tubes even in the absence of HPV target DNA. Additionally, DNA was thought to be retained via interactions between the charged DNA backbone and unreacted primary amines from the silanization step. Various blocking agents (e.g., non-fat milk, bovine serum albumin, glycerin, and synthetic urine) were tested to determine their effectiveness at reducing this background signal from undesired interactions. These studies showed that biotin (e.g., in milk) could saturate empty binding sites on the immobilized streptavidin, which could drastically reduce the interaction with HRP and therefore effectively block the glass surface from HRP absorption. Glycerin also was found to protect the unreacted amines from interacting with DNA, reducing the background. The final blocking buffer recipe therefore consisted of milk and TWEEN® to reduce protein interactions, and Denhardt's solution and glycerin to reduce DNA interactions. Together, these blocking agents protected the functionalized surface from interference and nonspecific absorption from a wide range of interactions.

Further refinement and optimization of the fabrication procedure was performed with the specific goal of increasing reproducibility. Toward that end, the reaction parameters were optimized for consistent, complete coverage of the glass surface. The adjusted parameters included reaction time (each step incubated for 10 minutes) and temperature (room temperature), which decreased variation between batches. A curing step that was added after the silanization provided a better monolayer surface. In addition, spacing of the capillaries with a clay manifold prevented the capillary tubes from touching during reaction. These adjustments resulted in increased repeatability, decreased reagent use and therefore production cost, decreased background signal, and increased positive signal intensity.

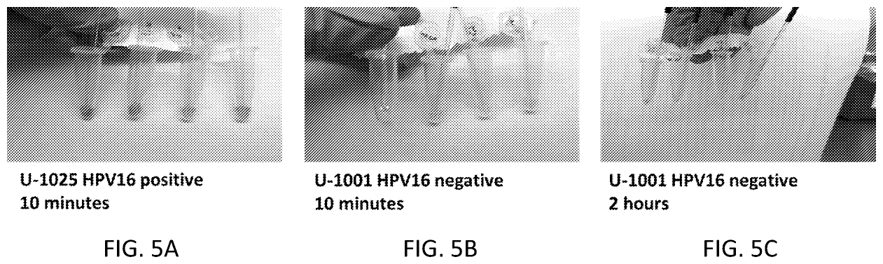

Glass capillary tubes (about 10 cm long with a diameter of about 2 mm) were fabricated with a rubber handle on one end and immobilized DNA probes on the other end. The tubes were successfully used with a diagnostic platform as described herein to detect of high-risk HPV strains in vitro and in patient urine. Samples from HPV16 positive and negative patients were readily distinguishable (FIGS. 5A-5C). Test results were verified to be stable for two hours at room temperature without color change, demonstrating the stability of the test result.

### Example 3—Refinement of Methods and Kit Components

Additional studies were conducted to stabilize HRP-DNA storage, improve batch-to-batch reproducibility, reduce the amount of streptavidin needed for fabrication, and improve the silanization film on the glass surface. Efforts were particularly focused on stability, as HRP-conjugate stability can significant impact the ability to obtain accurate results after extended periods of storage time.

HRP-DNA storage can be challenging. On one hand, the HRP structure can be damaged by repeated freeze-thaw cycles, likely due to ice crystal formation (Cao et al., Biotechnol Bioeng 82(6):684-690, 2003). On the other hand, DNA is susceptible to degradation if not frozen (e.g., at −20° C.). This can pose issues for the shelf stability of the kits provided herein.

The stability of the kits was analyzed and found to be short (weeks), as freshly made HRP-DNA solutions lost their ability to catalyze the TMB color changing reaction. This would make it difficult to store HRP-DNA long term, an issue that would translate to the final kits, necessitating specific storage conditions to ensure that they would not expire rapidly. To address this problem, parameters such as temperature, light, and buffer were tested. These studies revealed that this problem was caused mainly by repeated freeze-thaw cycles, and was dependent on HRP-DNA concentration. Addition of 2% polyethylene glycol (PEG) and storage at 4° C., without freezing, was shown to preserve the activity of the HRP conjugates. With these modifications, the HRP-DNA showed no decrease in catalytic ability even after months of storage.

As another means to improve stability, 1 μM HRP-DNA stock was prepared from an original 100 μM stock, and was frozen once. The 1 μM HRP-DNA stock was then removed from the freezer and thawed to make a 1×10⁻² μM working solution, after which the remaining 1 μM stock was kept in the refrigerator at 4° C., and not subjected to further freezing and thawing. This method solved most of the problems with reduced HRP activity, although the 1 μM HRP-DNA stock only lasted about two months before degradation of the DNA affected its ability to hybridize to target DNA, reducing the signal of the kit. This could significantly shorten the theoretical shelf life of the kit.

The above observations led to further investigation for a way to keep HRP-DNA in the freezer, in order to avoid DNA degradation while still maintaining the activity of HRP. In particular, 50% glycerol was added to the 1 μM HRP-DNA stock, which prevented the solution from freezing. Test results showed that even after 10 repeated freeze-thaw cycles, the HRP-DNA stock with 50% glycerol did not suffer significant HRP activity loss, while a corresponding control suffered a significant loss of activity. The effect of glycerol on DNA hybridization and HRP signal intensity was evaluated, revealing that leftover glycerol did not interfere with DNA hybridization in the 1×10⁻² μM working solution. In addition, HRP activity is not affected by the addition of glycerol. Thus, the addition of 50% glycerol to the HRP-DNA solution was identified as another means for improving stability and increasing the shelf life of the kit.

Additional work was done to ascertain how to maintain the activity of low concentrations of HRP-DNA, since low concentrations of HRP-DNA (1×10⁻² μM) lost activity much faster than high concentrations of HRP-DNA(1 μM). To address this problem, 2% polyethylene glycol (PEG) was added to the HRP-DNA working solution, resulting in significantly improved stability. However, the low concentration HRP-DNA still slowly lost activity, possibly due to structural changes in the protein caused by leaching of calcium ions (Chattopadhyay and Mazumdar, Biochem 39(1):263-270, 2000). This is one of the main causes of the lack of batch-to-batch reproducibility. Further studies are conducted to determine whether adding different levels of calcium chloride to the storage solution can shift the equilibrium to the intact HRP side while not inducing accelerated DNA degradation.

Another observed challenge related to stability was that the streptavidin-modified tubes were not able to immobilize biotinylated DNA after 2 to 3 weeks. After further study, it was determined that this was caused by streptavidin undergoing a non-reversible loss of water within its tertiary structure during long term storage. This problem was solved by adding a trehalose solution before drying. The trehalose replaced the role of water in stabilizing the streptavidin tertiary structure with hydrogen bonds.

In addition, studies were carried out in an attempt to reduce the amount of streptavidin used during fabrication of the elongate supports used in the test kits provided herein. As streptavidin is a protein, it is susceptible to denaturation. In addition, streptavidin is the limiting reagent in the fabrication process, as it is the most expensive and the most frequently ordered supply. A series of quantitative analyses using fluorescence microscopy were carried out to test how much streptavidin was really consumed in the fabrication, and to assess whether the amount of streptavidin used in the fabrication could be reduced. These studies revealed that less than 10% of streptavidin was consumed during each fabrication process, meaning that a significant amount of streptavidin was discarded after each experiment. However, it also was found that the concentration of streptavidin could not be significantly reduced, because even though it was not consumed in the reaction, reduced concentrations of streptavidin led to reduced coverage on the capillary tubes due to the kinetics of the reaction. Eventually, it was decided to use each streptavidin solution twice in fabrication, by placing half of each batch of capillary tubes in the solution and then replacing them with the other half of the tubes after the first reaction was complete. In this manner, only half of the original streptavidin was used while maintaining the same reaction rate and coverage. Test results showed that this change did not affect the immobilized amount of streptavidin on the glass surface. It is noted that it might be possible to use the streptavidin more than twice, which would improve the efficiency of streptavidin even more.

A series of tests also was conducted to evaluate hydrolysis of the aminosilane film on the glass surface. DNA probes typically were attached to the glass surface via an APTES silanization film. However, the APTES film will be depleted if it is immersed in water for long period of time, due to hydrolysis of the silane bond in aqueous solution. Studies were conducted to determine whether this problem might be resolved by reducing the time for which the tubes were exposed to aqueous solution during fabrication, using higher temperature and longer reaction time, using strict water-free methods, changing the silanization agent from APTES to AEAPTES, and changing the glass surface to a charge neutral hydrophobic surface but with covalent addition of covalent moieties. Reducing the reaction time from 1 hour to 30 minutes for each step in the fabrication process that involved an aqueous solution resulted in reduced hydrolysis of the silane bond, without any significant decrease in biotin, streptavidin, or DNA immobilization. No improvement was observed when AEAPTES was used instead of APTES, and no significant change was seen when the glass surface was coated to be more hydrophobic. Thus, reduced reaction times seemed to create more stable immobilized layers and reduced the time it took to fabricate the kits.

### Example 4—HRP-DNA Long-Term Storage Test

Studies were carried out to assess whether a viable method could be developed for storing HRP-DNA for a long period of time (more than 6 months). For example, tests were conducted to assess the long-term storage potential of the tests—particularly the DNA and HRP—at room temperature. Trials were performed with three DNA storage recipes, including storage in two different buffer solutions composed of Na₃PO₄, NaCl, ethanol, and CaCl₂, and dry-storage in a sugar matrix composed of trehalose and polyvinyl alcohol. Samples were stored at room temperature for two weeks, two months, or four months, and the quality of the DNA was assessed by TapeStation. These studies showed no significant difference between the quality of the stored DNA as compared to the original sample.

It is noted, however, that HRP typically requires being refrigerated for a maximum of 2 weeks without significant loss of activity. Based on previous results, it was apparent that HRP-DNA working solution (1×10⁻² μM) kept at 4° C. generally lost its specificity after one month, likely due to DNA degradation. A challenge for HRP-DNA long-term storage is that while long-term storage of DNA in the freezer is best (typically at −15° C.), HRP undergoes irreversible damage during the freeze-thaw cycles. Thus, HRP-DNA working solution is usually kept at 4° C. (2% PEG, 1× PBS solution). However, this temperature does not prevent DNA degradation during long-term storage. The following studies indicated that 2% ethanol at 4° C. was the best method for HRP-DNA long-term storage.

Because the preferred storage conditions for DNA and HRP are not compatible, three different methods were tested: vacuum drying (the original state of HRP-DNA as received from IDT), liquid storage at −15° C. (DNA preferred condition), and liquid storage at 4° C. (HRP preferred condition).

Samples tested in the vacuum drying method included the HRP-DNA working solution, as well as the working solution with added sucrose, trehalose, and polyvinyl alcohol (PVA) from several different recipes that are supposed to protect HRP during the drying process (Ivanova and Kuzmina, Mol Ecol Resources, 13(5):890-898, 2013). After drying the HRP-DNA at room temperature overnight, the samples were rehydrated and activity was tested, revealing that the activity of HRP in all samples was significantly decreased. It is possible that during the drying process, the salts in the solution (from PBS) precipitated out and caused some damage to the HRP protein structure. This approach may still have promise; additional studies are conducted to optimize the solvent recipe for HRP-DNA and lower the ionic strength significantly.

The −15° C. storage method was tested because a potential mechanism for HRP damage during freeze-thaw cycles is that ice crystals may irreversibly damage the protein structure of HRP. If the HRP-DNA solution is prevented from freezing, the HRP should remain intact despite the low temperature and the DNA can should be protected. In these tests, trehalose (1% and 5%), glycerol (1%, 10%, and 50%), and PEG (10% and 50%) were added to 0.5 μM HRP-DNA solution. Among all the different recipes tested, only glycerol at 50% was able to prevent HRP-DNA from being completely frozen. However, the activity of HRP was totally gone in the 50% glycerol sample after 6 months. It is possible that mechanisms such as equilibrium issues with the ion exchange in the solution caused the loss of activity.

Finally, the 4° C. storage method was tested by adding 2% ethanol, 2% polyethylene glycol (PEG), and 1× PBS buffer to the 1×10⁻² μM HRP-DNA solution. An advantage of this method is that the HRP-DNA working solution was ready to use. This method did not require rehydration of the HRP-DNA like the vacuum dried method, nor did it require dilution of the HRP-DNA like the −15° C. storage method. The ethanol is thought to serve as a radical scavenger to minimize DNA damage. The method was adapted from a protocol described elsewhere (Anchordoquy and Molina, Cell Preservation Technol, 5(4):180-188, 2007), and was modified in that EDTA was not added to the sample because it would likely damage HRP by removing the metal ion from its porphyrin structure. In addition, metal contamination was minimized by using nanopure water, clean labware and sterilized lab space. After 4 months of storage, HRP activity remained very high (FIG. 6), and the DNA appeared to be well maintained.

### Example 5—Refining the Glass Surface Modification Procedure

A new method for modifying the glass surface for use in DNA-directed immobilization techniques was developed in order to reduce or eliminate nonspecific absorption, thus significantly lowering the detection limit. Probes immobilized on a glass surface via biotin-NHS or streptavidin can be used in assays for detecting RNA, DNA, proteins, antibodies, or hormones, for example. The advantages of such techniques include their sensitivity and selectivity due to specific recognition between DNA or proteins and the high selectivity and stable bond between biotin and streptavidin. In some cases, immobilization can be achieved by attachment of biotin-NHS to an aminosilanized glass surface via hydroxyl groups on the glass surface. The glass surface can then be used as biotinylated glass or can be further modified to immobilize a layer of streptavidin, to which biotinylated DNA or biotin-conjugated protein can be attached and used to specifically detect target biomarkers. In some cases, streptavidin can be attached to the aminosilanized glass surface, in which case a biotin-DNA conjugate can be used as a capturing probe. Due to practical limits, there may be a significant amount of unreacted APTES on the glass surface, which can lead to high background signal due to nonspecific absorption. Further, the Si-O bond that binds to the glass surface can be susceptible to water hydrolysis. This means that when in contact with water, the already immobilized biotin-NHS and/or streptavidin can be slowly depleted, which may lead to loss of surface streptavidin capacity in a matter of hours.

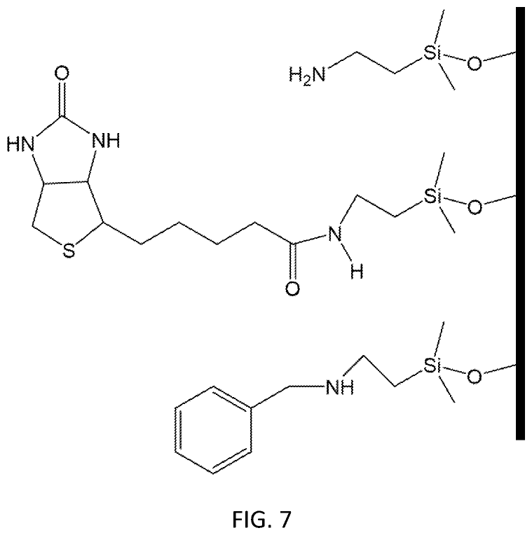

In the present studies, a covalently bound benzaldehyde coating was added after the biotin-NHS step (FIG. 7, middle). This addition replaced the primary amine (FIG. 7, top), which was protonated at physiological pH and thus labile, with an inert and hydrophobic benzene ring (FIG. 7, bottom). Modification with a benzaldehyde coating can eliminate nonspecific adsorption of DNA and protein, and also can reduce the loss of surface streptavidin capacity due to hydrolysis. The benzaldehyde coating can be stable and inert, and not readily washed away or displaced by other species. The hydrophobic layer also can protect immobilized species from hydrolysis in water, allowing for longer shelf life and better stability in non-ideal conditions when users do not follow instructions well. In addition, the chemical modification to the glass can be performed early in the fabrication process and provides benefits that would otherwise need to be applied during or even after use with a sample. This means that assay kits can be developed such that everyday users can self-administer diagnostic kits without the need for expensive equipment, hazardous chemicals, or the aid of trained professionals—much like a home pregnancy kit.

### Example 6—Improving Stability, Efficiency, and Sensitivity

The studies described above indicated that long term storage of HRP-DNA was best achieved at 4° C. with 2% ethanol, 2% PEG, and 1×PBS buffer, although vacuum drying also has some merit. Further studies are conducted to evaluate the effectiveness of lower amounts of ethanol, and to determine whether the 2% ethanol recipe allows HRP-DNA and other DNA to be maintained at room temperature for long periods of time. This makes the test kit easier to use in the field, as refrigeration would not be required.

To determine whether the efficiency and the sensitivity of the test can be further improved, several areas are investigated. First, a high-quality APTES monolayer is applied to the tip of the glass capillary tube. Because this improvement reduces hydrolysis can lead to loss of immobilized DNA, it increases batch reproducibility and reduces DNA non-specific adsorption, which reduces background noise. Second, to prevent denatured DNA from being renatured to itself, which can decrease hybridization efficiency, blocking oligonucleotides are designed for specific target strains. Such blocking oligonucleotides may increase accessibility of the target binding site, allowing it to be captured or labeled. Third, to improve target capturing efficiency and thus detection sensitivity, work is done to increase the capture area using, for example, glass microbeads or magnetic beads that can be easily dispersed throughout the sample and can aggregate due to gravity, without the need for additional tools.

### Example 7—Rapid Point-of-Care Detection of COVID-19

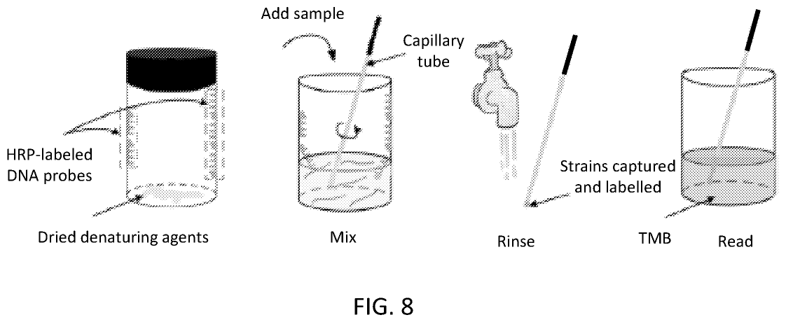

Testing for detection of COVID-19 infection is limited by the availability of supplies and reagents, distribution chains, and laboratory processing time. As described above, a novel real-time and low-cost rapid point-of-care detection platform was developed for targets such as HPV and influenza strains, where a sample can be tested and interpreted by the end user using a fluid sample (e.g., saliva or urine). The test is scaled up to mass production with relative ease. In some cases, the test is based on dipping a molecularly modified glass capillary tube into a sample, then mixing, rinsing, and reading as depicted in FIG. 8. A positive result leads to a color change easily distinguishable by the naked eye, and the turnaround time for the test is short (typically on the order of 30 minutes or less).

A test as described herein is modified to generate a platform for detecting COVID-19 by constructing SARS-CoV-2 probes based on qPCR probes made available by the CDC. The design is tested in vitro with positive (COVID-19) and negative (MERS and SARS) control plasmids, which are available from Integrated DNA Technologies (Coralville, IA). After a comparable test performance to the CDC qPCR recommended test is achieved, patient samples are tested and results are compared to a clinical report. The target population for enrollment in this study includes, for example, people who have had contact with a confirmed COVID-19 positive case, to capture a larger percentage of positive cases. Completion of this work enables point-of-care self-sampling of suspected COVID-19 patients, interpretable in real-time by the end user. The test reduces the need for healthcare workers to directly sample subjects, thus minimizing the exposure of health care workers and reducing use of personal protective equipment. The test also alleviates the burden to clinical laboratories to process the tests, and substantially reduces the turnaround time from testing to result.

The fabrication process is optimized to improve reproducibility in color intensity between replicates and to further reduce background signals. In addition, a more integrated test that further minimizes user steps is developed. To adapt the test for COVID-19, probe sequences are based on SARS-CoV-2 PCR probe sequences (TABLE 2).

TABLE 2
Target probes for COVID-19

| CDC probe | COView Probe |
|---|---|
| 2019-nCoV_N1-P | 5′-HRP-ACCCCGCATTACGTTTGGTGGACC-Biotin-3′ |
| | (SEQ ID NO: 29) |
| 2019-nCoV_N2-P | 5′-HRP-ACAATTTGCCCCCAGCGCTTCAG-Biotin-3′ |
| | (SEQ ID NO: 30) |
| 2019-nCoV_N3-P | 5′-HRP-AYCACATTGGCACCCGCAATCCTG-Biotin-3′ |
| | (SEQ ID NO: 31) |
| RP-P | 5′-HRP-TTCTGACCTGAAGGCTCTGCGCG-Biotin-3′ |
| | (SEQ ID NO: 32) |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An elongate support comprising a first surface, a first nucleic acid immobilized on the first surface, and a second nucleic acid reversibly attached to the first surface or to a second surface, wherein the first surface is silanized, wherein the first nucleic acid is complementary to a first segment of a selected nucleic acid marker, and wherein the second nucleic acid is complementary to a second segment of the selected nucleic acid marker.

2. The elongate support of claim 1, wherein the elongate support comprises glass.

3. The elongate support of claim 1 or claim 2, wherein the elongate support is a glass rod.

4. The elongate support of claim 3, wherein the glass rod is a capillary tube.

5. The elongate support tube of any one of claims 1 to 4, wherein the first nucleic acid is immobilized on the first surface via biotin-streptavidin coupling.

6. The elongate support of any one of claims 1 to 5, wherein the second nucleic acid is dry-stored on the first surface or the second surface.

7. The elongate support of any one of claims 1 to 6, wherein the second nucleic acid is coupled to a means for visual detection.

8. The elongate support of claim 7, wherein the means for visual detection is horseradish peroxidase (HRP).

9. The elongate support of any one of claims 1 to 8, wherein the marker is from one or more high-risk human papillomavirus (HPV) strains.

10. The elongate support of claim 9, wherein the one or more high-risk HPV strains comprise one or more of HPV 16, HPV 18, HPV 31, HPV 33, HPV 35, HPV 39, HPV HPV 51, HPV 52, HPV 56, HPV 58, HPV 59, and HPV 68.

11. The elongate support of any one of claims 1 to 8, wherein the marker is from an infectious agent.

12. The elongate support tube of claim 11, wherein the infectious agent is an influenza virus.

13. The elongate support of claim 11, wherein the infectious agent is Escherichia coli.

14. The elongate support of any one of claims 1 to 13, wherein an external surface of the support comprises a coating or handle.

15. The elongate support of any one of claims 1 to 14, wherein the silanized first surface comprises functional groups derived from (3-aminopropyl)triethoxysilane (APTES), N-(2-amionethyl)-3-aminopropyltriethoxysilane (AEAPTES), N-(2-aminoethyl)-3-aminopropyltrimethoxysilane (AEAPTMS), or N-(6-aminohexyl)amionmethyltriethoxysilane (AHAMTES).

16. The elongate support of any one of claims 1 to 15, wherein the silanized first surface comprises benzaldehyde-protected silane groups.

17. An elongate support comprising a silanized surface with a nucleic acid immobilized thereon, wherein the nucleic acid is complementary to a first segment of a selected nucleic acid marker.

18. The elongate support of claim 17, wherein the support comprises glass.

19. The elongate support of claim 17 or claim 18, wherein the elongate support is a glass rod.

20. The elongate support of claim 19, wherein the glass rod is a capillary tube.

21. The elongate support of any one of claims 17 to 20, wherein the nucleic acid is immobilized on the silanized surface via biotin-streptavidin coupling.

22. The elongate support of any one of claims 17 to 21, wherein the marker is from one or more high-risk HPV strains.

23. The elongate support of claim 22, wherein the one or more high-risk HPV strains comprise one or more of HPV 16, HPV 18, HPV 31, HPV 33, HPV 35, HPV 39, HPV HPV 51, HPV 52, HPV 56, HPV 58, HPV 59, and HPV 68.

24. The elongate support of any one of claims 17 to 23 wherein the marker is from an infectious agent.

25. The elongate support of claim 24, wherein the infectious agent is an influenza virus.

26. The elongate support of claim 24, wherein the infectious agent is Escherichia coli.

27. The elongate support of any one of claims 17 to 26, wherein an external surface of the support comprises a coating or handle.

28. The elongate support of any one of claims 17 to 27, wherein the silanized surface comprises functional groups derived from APTES, AEAPTES, AEAPTMS, or AHAMTES.

29. The elongate support of any one of claims 17 to 27, wherein the silanized surface comprises benzaldehyde-protected silane groups.

30. A kit comprising:
(a) an elongate support comprising a first surface, a first nucleic acid immobilized on the first surface, and a second nucleic acid reversibly attached to the first surface or to a second surface, wherein the first surface is silanized, wherein the first nucleic acid is complementary to a first segment of a selected nucleic acid marker, and wherein the second nucleic acid is complementary to a second segment of the selected nucleic acid marker and is labeled with a means for visual detection;
(b) a receptacle for receiving a biological fluid sample; and
(c) a vessel containing a substrate that interacts with the means for visual detection.

31. The kit of claim 30, wherein the first nucleic acid is immobilized on the first surface via biotin-streptavidin coupling.

32. The kit of claim 30 or claim 31, wherein the second nucleic acid is dry-stored on the first surface or the second surface.

33. The kit of any one of claims 30 to 32, wherein the means for visual detection is HRP.

34. The kit of any one of claims 30 to 33, wherein the marker is from one or more high-risk HPV strains.

35. The kit of claim 34, wherein the one or more high-risk HPV strains comprise one or more of HPV 16, HPV 18, HPV 31, HPV 33, HPV 35, HPV 39, HPV 45, HPV 51, HPV 52, HPV 56, HPV 58, HPV 59, and HPV 68.

36. The kit of any one of claims 30 to 33, wherein the marker is from an infectious agent.

37. The kit of claim 36, wherein the infectious agent is an influenza virus.

38. The kit of claim 36, wherein the infectious agent is Escherichia coli.

39. The kit of any one of claims 30 to 38, wherein the substrate is tetramethylbenzidine (TMB).

40. The kit of any one of claims 30 to 39, wherein an external surface of the elongate support comprises a coating or handle.

41. The kit of any one of claims 30 to 40, wherein the silanized surface comprises functional groups derived from APTES, AEAPTES, AEAPTMS, or AHAMTES.

42. The kit of any one of claims 30 to 41, wherein the silanized first surface comprises benzaldehyde-protected silane groups.

43. The kit of any one of claims 30 to 42, wherein the elongate support comprises glass.

44. The kit of any one of claims 30 to 43, wherein the elongate support is a glass rod.

45. The kit of any one of claims 30 to 44, wherein the elongate support is a glass capillary tube.

46. A kit comprising:
(a) an elongate support comprising a silanized surface with a first nucleic acid immobilized thereon, wherein the first nucleic acid is complementary to a first segment of a selected nucleic acid marker;
(b) a receptacle for receiving a biological fluid sample;
(c) a first vessel containing a second nucleic acid that is complementary to a second segment of the selected nucleic acid marker, wherein the second nucleic acid is labeled with a means for visual detection; and
(d) a second vessel containing a substrate that interacts with the means for visual detection.

47. The kit of claim 46, wherein the first nucleic acid is immobilized on the silanized surface via biotin-streptavidin coupling.

48. The kit of claim 46 or claim 47, wherein the means for visual detection is HRP.

49. The kit of any one of claims 46 to 48, wherein the marker is from one or more high-risk HPV strains.

50. The kit of claim 49, wherein the one or more high-risk HPV strains comprise one or more of HPV 16, HPV 18, HPV 31, HPV 33, HPV 35, HPV 39, HPV 45, HPV 51, HPV 52, HPV 56, HPV 58, HPV 59, and HPV 68.

51. The kit of any one of claims 46 to 48, wherein the marker is from an infectious agent.

52. The kit of claim 51, wherein the infectious agent is an influenza virus.

53. The kit of claim 51, wherein the infectious agent is Escherichia coli.

54. The kit of any one of claims 46 to 53, wherein the first vessel contains the second nucleic acid in a fluid that comprises 2% polyethylene glycol (PEG).

55. The kit of any one of claims 46 to 54, wherein the substrate is TMB.

56. The kit of any one of claims 46 to 55, wherein an external surface of the elongate support comprises a coating or handle.

57. The kit of any one of claims 46 to 56, wherein the silanized surface comprises functional groups derived from APTES, AEAPTES, AEAPTMS, or AHAMTES.

58. The kit of any one of claims 46 to 57, wherein the silanized surface comprises benzaldehyde-protected silane groups.

59. The kit of any one of claims 46 to 58, wherein the elongate support comprises glass.

60. The kit of any one of claims 46 to 59, wherein the elongate support is a glass rod.

61. The kit of any one of claims 46 to 60, wherein the elongate support is a capillary tube.

62. A method for determining that a biological fluid contains a selected marker, wherein the method comprises:
(a) providing an elongate support comprising a first surface, a first nucleic acid immobilized on the first surface, and a second nucleic acid reversibly attached to the first surface or to a second surface, wherein the first surface is silanized, wherein the first nucleic acid is complementary to a first segment of a selected nucleic acid marker, and wherein the second nucleic acid is complementary to a second segment of the selected nucleic acid marker and is labeled with a means for visual detection;
(b) placing at least a portion of the elongate support into a vessel containing a biological fluid, such that the first or first and second surfaces are contacted by the biological fluid;
(c) removing the elongate support from the vessel and rinsing the elongate support;
(d) placing at least a portion of the elongate support into a vessel containing a substrate that interacts with the means for visual detection, such that the first surface is contacted by the substrate; and
(e) visually inspecting the vessel containing the substrate, or a sample of the substrate, to determine that the signal is present, thus indicating the presence of the selected marker in the biological fluid.

63. The method of claim 62, wherein the elongate support comprises glass.

64. The method of claim 62 or claim 63, wherein the elongate support is a glass rod.

65. The method of claim 64, wherein the glass rod is a capillary tube.

66. The method of any one of claims 62 to 65, wherein the first nucleic acid is immobilized on the first surface via biotin-streptavidin coupling.

67. The method of any one of claims 62 to 66, wherein the second nucleic acid is dry-stored on the first surface or the second surface.

68. The method of any one of claims 62 to 67, wherein the second nucleic acid is coupled to a means for visual detection.

69. The method of claim 68, wherein the means for visual detection is HRP.

70. The method of any one of claims 62 to 69, wherein the marker is from one or more high-risk HPV strains.

71. The method of claim 70, wherein the one or more high-risk HPV strains comprise one or more of HPV 16, HPV 18, HPV 31, HPV 33, HPV 35, HPV 39, HPV 45, HPV 51, HPV 52, HPV 56, HPV 58, HPV 59, and HPV 68.

72. The method of any one of claims 62 to 71, wherein the marker is from an infectious agent.

73. The method of claim 72, wherein the infectious agent is an influenza virus.

74. The method of claim 72, wherein the infectious agent is Escherichia coli.

75. The method of any one of claims 62 to 74, wherein an external surface of the elongate support comprises a coating or handle.

76. The method of any one of claims 62 to 75, wherein the silanized first surface comprises functional groups derived from APTES, AEAPTES, AEAPTMS, or AHAMTES.

77. The method of any one of claims 62 to 76, wherein the silanized first surface comprises benzaldehyde-protected silane groups.

78. A method for determining that a biological fluid contains a selected marker, wherein the method comprises:
(a) providing an elongate support comprising a silanized surface with a first nucleic acid immobilized thereon, wherein the first nucleic acid is complementary to a first segment of a selected nucleic acid marker;
(b) placing at least a portion of the elongate support into a first vessel containing a biological fluid, such that the silanized surface is contacted by the biological fluid;
(c) removing the elongate support from the first vessel and rinsing the elongate support;
(d) placing at least a portion of the elongate support into a second vessel comprising a second nucleic acid that is complementary to a second segment of the selected nucleic acid marker and is labeled with a means for visual detection, such that the silanized surface is contacted by the second nucleic acid;
(e) removing the elongate support from the second vessel and rinsing the elongate support;
(f) placing at least a portion of the elongate support into a third vessel containing a substrate that interacts with the means for visual detection, such that the silanized surface is contacted by the substrate; and
(g) visually inspecting the third vessel containing the substrate, or a sample of the substrate, to determine that the signal is present, thus indicating the presence of the selected marker in the biological fluid.

79. The method of claim 78, wherein the elongate support comprises glass.

80. The method of claim 78 or claim 79, wherein the elongate support is a glass rod.

81. The method of claim 80, wherein the glass rod is a capillary tube.

82. The method of any one of claims 78 to 81, wherein the nucleic acid is immobilized on the silanized surface via biotin-streptavidin coupling.

83. The method of any one of claims 78 to 82, wherein the marker is from one or more high-risk HPV strains.

84. The method of claim 83, wherein the one or more high-risk HPV strains comprise one or more of HPV 16, HPV 18, HPV 31, HPV 33, HPV 35, HPV 39, HPV 45, HPV 51, HPV 52, HPV 56, HPV 58, HPV 59, and HPV 68.

85. The method of any one of claims 78 to 84 wherein the marker is from an infectious agent.

86. The method of claim 85, wherein the infectious agent is an influenza virus.

87. The method of claim 85, wherein the infectious agent is Escherichia coli.

88. The method of any one of claims 78 to 87, wherein an external surface of the elongate support comprises a coating or handle.

89. The method of any one of claims 78 to 88, wherein the silanized surface comprises functional groups derived from APTES, AEAPTES, AEAPTMS, or AHAMTES.

90. The method of any one of claims 78 to 89, wherein the silanized surface comprises benzaldehyde-protected silane groups.

91. A method for preparing a silanized glass surface, wherein the method comprises:
reacting a glass surface with a silane-containing compound,
coupling an amino group of the silane-containing compound to biotin-N-hydroxysuccinimide (biotin-NHS) to yield a silanized, biotinylated surface, and
reacting the silanized, biotinylated surface with benzaldehyde.


SEQUENCE LISTING (32 sequences)

SEQ ID NO: 1 - DNA, 28 bp, Artificial Sequence
  synthetic oligonucleotide
  misc_feature (13)..(13): n = fluorescein-dT
  misc_feature (14)..(14): n = riboadenosine
  misc_feature (15)..(15): n = dabcyl-dT
actcttcctagcnnnggttcgatcaaga

SEQ ID NO: 2 - DNA, 99 bp, Artificial Sequence
  synthetic oligonucleotide
cacggatcctgacaaggatgtgtgcgttgtcgagacctgcgaccggaacactacactgtgtgggatggatttctttacagttgtgtgcagctccgtccg

SEQ ID NO: 3 - DNA, 452 bp, Human papillomavirus type 16
gcacagggacataataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaacctcccccaggaggcacactagaagatacttataggtttgtaacccaggcaattgcttgtcaaaaacatacacctccagcacctaaagaagatgatccccttaaaaaatacactttttgggaagtaaatttaaaggaaaagttttctgcagacctagatcagtttccttttggacg

SEQ ID NO: 4 - DNA, 19 bp, Escherichia coli
acggcgttactatcctctc

SEQ ID NO: 5 - DNA, 21 bp, Escherichia coli
tctttcccctcttttagtcag

SEQ ID NO: 6 - DNA, 20 bp, Escherichia coli
ttcacctttccctcaggatg

SEQ ID NO: 7 - DNA, 20 bp, Escherichia coli
tccgttcggctaagtcagtt

SEQ ID NO: 8 - DNA, 18 bp, Escherichia coli
acctgcggcaagtcgttt

SEQ ID NO: 9 - DNA, 20 bp, Escherichia coli
cgccggttacacgtagtgat

SEQ ID NO: 10 - DNA, 20 bp, Escherichia coli
ccagtctatgccaggttgct

SEQ ID NO: 11 - DNA, 20 bp, Escherichia coli
ctagctttgccaccaccatt

SEQ ID NO: 12 - DNA, 24 bp, Escherichia coli
tcatgagcctgctggaagttatca

SEQ ID NO: 13 - DNA, 20 bp, Escherichia coli
agtggtggcagcgaaactat

SEQ ID NO: 14 - DNA, 18 bp, Escherichia coli
tccgctcccgttactcag

SEQ ID NO: 15 - DNA, 20 bp, Escherichia coli
gcttattctcccgcatcaaa

SEQ ID NO: 16 - DNA, 21 bp, Escherichia coli
ggagacgctgaatacaactga

SEQ ID NO: 17 - DNA, 20 bp, Escherichia coli
cggtgcgtttaacacagcta

SEQ ID NO: 18 - DNA, 20 bp, Escherichia coli
atccggattatcaagctcca

SEQ ID NO: 19 - DNA, 20 bp, Escherichia coli
ctgtgaatccagtttcgggt

SEQ ID NO: 20 - DNA, 19 bp, Escherichia coli
cgaaattggacaagcgatg

SEQ ID NO: 21 - DNA, 20 bp, Escherichia coli
aggtatccaaatccgcactg

SEQ ID NO: 22 - DNA, 20 bp, Escherichia coli
atccgtcaggtgtttgtggt

SEQ ID NO: 23 - DNA, 20 bp, Escherichia coli
gggactgccacaatgaattt

SEQ ID NO: 24 - DNA, 20 bp, Escherichia coli
tttgcaaccgacatctacca

SEQ ID NO: 25 - DNA, 20 bp, Escherichia coli
attggacaagcgtccaacac

SEQ ID NO: 26 - DNA, 20 bp, Escherichia coli
agaggatttctcacgccagg

SEQ ID NO: 27 - DNA, 20 bp, Escherichia coli
  misc_feature (3)..(3): M = A or C
  misc_feature (6)..(6): H = A or C or T
ggmathgaaattcgccactg

SEQ ID NO: 28 - DNA, 20 bp, Escherichia coli
gcaagttcattgaacagggt

SEQ ID NO: 29 - DNA, 24 bp, Severe acute respiratory syndrome coronavirus 2
accccgcattacgtttggtggacc

SEQ ID NO: 30 - DNA, 23 bp, Severe acute respiratory syndrome coronavirus 2
acaatttgcccccagcgcttcag

SEQ ID NO: 31 - DNA, 24 bp, Severe acute respiratory syndrome coronavirus 2
  misc_feature (2)..(2): Y = T or C
aycacattggcacccgcaatcctg

SEQ ID NO: 32 - DNA, 23 bp, Homo sapiens
ttctgacctgaaggctctgcgcg